(19) United States
(12) Patent Application Publication
(10) Pub. No.: US 2009/0264708 A1
(43) Pub. Date: Oct. 22, 2009

(54) SINGLE-USE MULTI-PLATFORM INTUBATION AND SURGICAL APPARATUS

(76) Inventors: Jack PACEY, Vancouver (CA); Mitchell Visser, Burnaby (CA)

Correspondence Address: BLACK LOWE & GRAHAM, PLLC, 701 FIFTH AVENUE, SUITE 4800, SEATTLE, WA, 98104, US

(21) Appl. No.: 12/372,626

(22) Filed: Feb. 17, 2009

Related U.S. Application Data
(60) Provisional application No. 61/029,268, filed on Feb. 15, 2008.

(51) Int. Cl. A61B 1/267

(52) U.S. Cl. 600/187; 600/188

(57) ABSTRACT

A multipurpose surgical platform and intubation apparatus for ENT or Surgery with a lifting bar handle, video scope passage and multiple operating channels is described. The surgical platform may have a number of configurations and methods of assembly having an operating stage to allow delivery of the relevant scopes and tools reliably and with an angulation that reduces the mechanical force required to obtain the access needed for diagnosis and therapy. Laser surgery, smoke evacuation and jet ventilation are design features provided by the single-use surgical platform and intubation apparatus.

### CROSS REFERENCES To RELATED APPLICATIONS

This application claims priority to and incorporates by reference in its entirety U.S. Provisional Patent Application No. 61/029,268 filed Feb. 15, 2008.

This application describes embodiments that can be usefully combined with, or used in conjunction with applicant's other inventions described in the following patents and co-pending applications, each and all of which are incorporated by reference in their entirety: U.S. Pat. No. 6,142,144 filed as U.S. patent application Ser. No. 09/060,891 on Apr. 15, 1998; U.S. Pat. No. 6,655,377 filed as U.S. patent application Ser. No. 10/356,705 on Jan. 30, 2003; U.S. patent application Ser. No. 11/285,743 filed Nov. 21, 2005; U.S. Provisional Patent Application No. 60/862,192 filed Oct. 19, 2006; U.S. patent application Ser. No. 11/645,086 filed Dec. 21, 2006; U.S. patent application Ser. No. 11/925,868 filed Oct. 27, 2007; U.S. Provisional Patent Application No. 61/027,377 filed Feb. 8, 2008; U.S. patent application Ser. No. 12/368,298 filed Feb. 9, 2009.

### FIELD OF THE INVENTION

The present invention is in the field of anesthesiology, and, in particular, devices and apparatuses that provide for both laryngoscopy and endotracheal intubation for anesthesia and for subsequent examination, surgery and/or other procedures to be performed on the upper airway of a patient.

### BACKGROUND OF THE INVENTION

Endotracheal intubation provides the current preferred method for control of the airway for mechanical ventilation. One goal of the intubation process is to locate the distal end of an endotracheal tube in the larynx with the proximal end outside the patient's mouth in order to establish an airway. A laryngoscope is inserted into the mouth of the patient so that the distal end of the instrument is located in the glottis, adjacent to the vocal cords. An endotracheal tube is slid through the instrument during or after insertion of the instrument.

Additionally, laryngoscopes are also used to enable a physician to observe and operate on structures of the airway and other portions of the neck and throat anatomy, all while the patient is under anesthesia. Historically, such access for observation and surgery is provided by use of a special purpose laryngoscope, such as the anterior commissure laryngoscope. The anterior commissure laryngoscope provides the physician with a direct line-of-sight view of the larynx for intubation and subsequent observation or surgery, such as laser surgery or biopsy. The anterior commissure laryngoscope, however, can require substantial mechanical force to straighten a patient's airway. This application of substantial force can injure patients.

Observation of or surgery on a patient's upper airway or other related structures of the anatomy can require the use of many different instruments, sometimes during the same procedure. For example, a physician might employ a flexible bronchoscope, including one with a laser capable of targeting and destroying tissue. The physician might need to supply light to enhance visibility of the region, might need to supply jet ventilation to the area during this procedure, and might need to remove smoke from burning tissue. The physician might need to introduce forceps or biopsy needles into the airway during a procedure. These are only examples of the kinds of instruments a physician might want to introduce into a patient's upper airway.

All laryngoscopes and/or their associated accessories come into direct contact with patient tissues and bodily fluids. Many laryngoscopes have restrictions on the methods that can be used to sterilize them due to having parts that can be damaged or destroyed by autoclaving or rough handling. Laryngoscopes therefore need to undergo time consuming, high level disinfection procedures before being re-used on subsequent patients. This leads to delay and reduced cost effectiveness. The advent of more intense use of medical equipment which encounters intimate contact with patients has led to the need to provide adaptable equipment that requires a minimum of down time.

### BRIEF DESCRIPTION OF THE DRAWINGS

Preferred and alternative examples of the present invention are described in detail below with reference to the following drawings:

FIG. 1 schematically depicts a U-shaped laryngoscope not having the design features or ergonomic advantages of the embodiments described in FIGS. 4-10B below;

FIG. 2 schematically depicts an operator deploying a fiberoptic bronchoscope while using the U-shaped laryngoscope of FIG. 1;

FIG. 3 schematically depicts a an operator deploying a laser surgical apparatus while using the U-shaped laryngoscope of FIG. 1;

FIG. 4 schematically depicts an auxiliary support arm or scaffold structure holding a transparent laryngoscope with a channel for passage of instruments and a flexible bronchoscope passing through that channel;

FIG. 5 illustrates the angle of inclination between the entrance and exit ports 31 and 35 of an embodiment of the transparent laryngoscope 12;

FIG. 6 schematically depicts an operator controlling a bronchoscope passing through a channel of the transparent laryngoscope;

FIGS. 7A and 7B schematically depict a perspective side view of the tongue blade region of the transparent laryngoscope having different lengths and curvatures and showing a bronchoscope emerging from the distal port of the channel and protruding beneath the tongue blade region;

FIG. 7C illustrates a particular embodiment of the end of a fiber optic cable emerging beneath the tongue blade region of the embodiments illustrated in FIGS. 7A and 7B;

FIG. 8 schematically depicts a bottom view of the blade region depicted in FIGS. 7A and 7B;

FIGS. 9A-B schematically depict an embodiment of the laryngoscope including a suction channel;

FIGS. 10A-B schematically depict alternate embodiment of the transparent laryngoscope having a single use shell, wire cover, and an HDTV digital core;

FIG. 11 illustrates a lens canopy for a transparent laryngoscope; and

FIG. 12 illustrates another embodiment for a lens canopy of a transparent laryngoscope.

### DETAILED DESCRIPTION OF THE PREFERRED EMBODIMENTS

A multipurpose, single-use, observational and surgical platform adapted for intubation procedures and for subsequent examination and/or surgery in the upper airway and related anatomy (ear-nose-throat or ENT). The observational and surgical platform includes a substantially transparent laryngoscope which can include a channel for a camera or other viewing device to assist the operator in positioning the laryngoscope without requiring excessive or undesirable manipulation of the patient's head, neck, and other anatomy.

The laryngoscope is further equipped with at least one channel through which an operator can pass instruments for observation and/or surgery of the upper airway. During use, the channel's entry port remains outside the patient so that the operator can insert an instrument into it, while the exit port is nearer the distal end of the laryngoscope and the portion of the patient's anatomy of interest to the operator. In a preferred embodiment, the entry and exit ports of the channel are inclined to one another at a partially curved and/or an obtuse angle. The channel also preferably is configured to be able to convey a fiber optic for visual observation or delivery of therapeutic laser applications to a patient's region-of-interest by users operating the fiber optic through the transparent laryngoscope in an ergonomic manner.

The substantially transparent laryngoscope can include multiple channels, one or more of which can be visible to the operator so that the operator can visually confirm routing instruments into and through the channel or channels within the laryngoscope. In these embodiments, various instruments such as, for example, viewing optics, surgical optics and vacuum instruments may be conveyable or conveyed in separate visible channels. In yet a further embodiment, multiple instruments can be conveyable or conveyed in a single channel which has a single entrance and exit port but includes a mid-channel divider or midline to route the separate instruments.

The transparent laryngoscope can be positionable by an auxiliary support arm or scaffold structure that has moveable components to allow the operator to secure the laryngoscope in a particular position. In this manner, the operator can re-position the laryngoscope as desired to insure the correct delivery of instruments to the patient's anatomy while also enhancing the operator's own ease of use and position during use of the surgical platform.

The surgical platform includes a number of laryngoscope configurations and methods of assembly which allow reliable delivery of the relevant observation and surgical instruments through the laryngoscope at angles which reduce the mechanical forces required to obtain the access needed for diagnosis and therapy. Instruments for various procedures, such as Laser surgery, smoke evacuation and jet ventilation, can be conveyed to the patient's anatomy through or alongside embodiments of the transparent laryngoscope. This provides a single-use, multi-purpose surgical platform and intubation apparatus for ENT procedures. Other embodiments described below also provide for a lifting bar handle, video scope passages and multiple operating channels.

Embodiments disclosed herein include a single use intubation apparatus for placing an endotracheal tube by an operator using a substantially transparent laryngoscope having a curved blade, a handle, and a middle section between the blade and handle. the transparent laryngoscope has at least one channel routed through it with a first port located on the handle and a second port located in the blade, the first and second port inclined to one another at an obtuse angle. Observational and/or surgical instruments such as, for example, a fiberoptic bronchoscope, a therapeutic laser, a instrument for jet ventilation, and/or a vacuum source, can be passed into the patient's throat by passing the instrument into first port, through the channel, and out the second port. A light source may be removeably attachable to the fiber optic cable that emerges from the second port located near or beneath the blade. A viewing device may be removeably attachable to the end of the fiber optic cable entering the first port, so that light when conveyed from the light source to the region of interest via the at least one first channel provides a view of the region of interest via the viewing device. Other embodiments provide for a scaffolding structure that may securely hold and reposition the substantially transparent laryngoscope to form a surgical platform so that observation and/or surgery may be undertaken while the device remains securely positioned.

Other embodiments provide for an option for jet ventilation, such as, for example, using a laser aimed jet ventilation catheter which has a fiber optic portion which may be central or peripheral and conducts light down the catheter in such a way that the light exits at the tip and serves as a visual guide for the anesthesiologist to aim the jet past the vocal cords in a way that will not entrain particles of tissue from the operating site but will provide air flow and pressure increase in the trachea and enable oxygenation and ventilation.

The ENT surgical platform allows laser treatment to be conducted using a dedicated therapeutic laser fiber-optic which passes through a centrally disposed channel in the laryngoscope. The surgical platform allows an operator to positively control the laser's position for treatment of a patient's anatomy. The centrally disposed channel is angled to conform to the patient's airway anatomy.

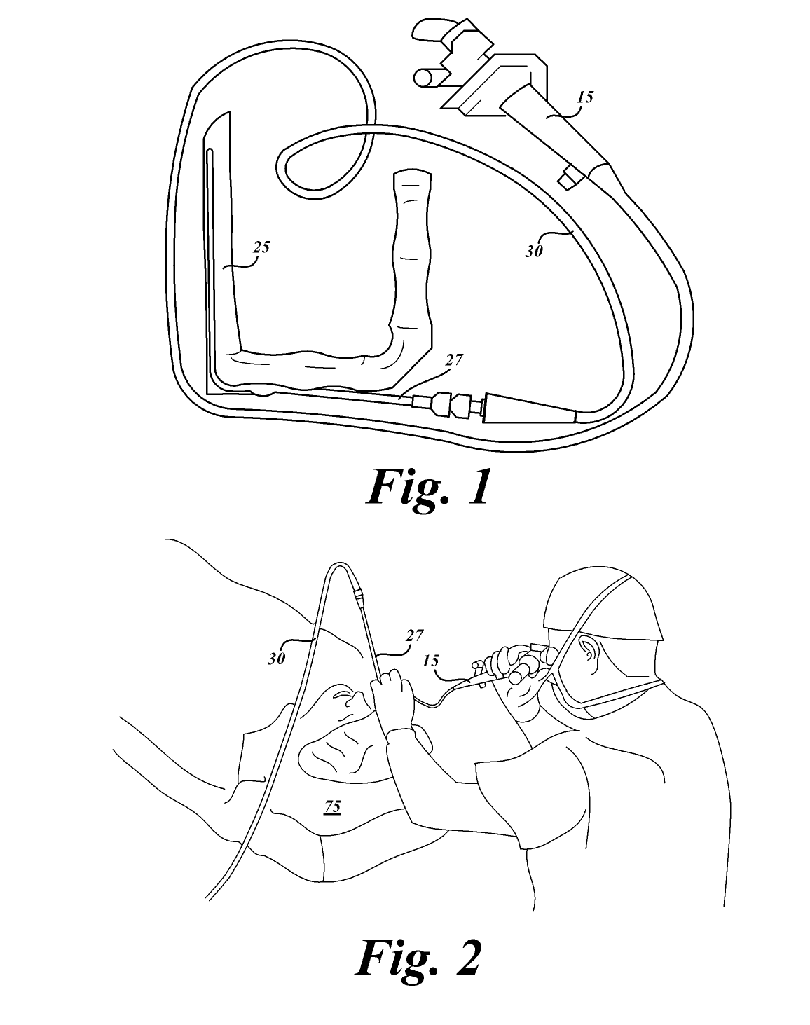

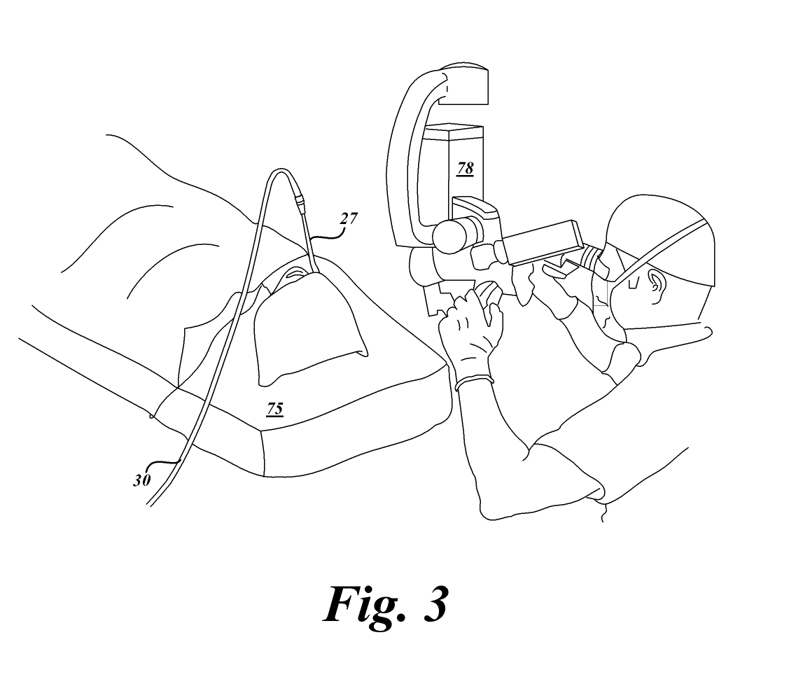

Many U-shaped laryngoscope designs as shown in FIG. 1 are found by some medical practitioners to be unwieldy to use in insertion and placing breathing tubes into the airway of patients. Moreover, viewing procedures conducted by medical staff as depicted in FIGS. 2 and 3 are found to be ergonomically difficult in that the medical staff has to crouch down to the patient's level.

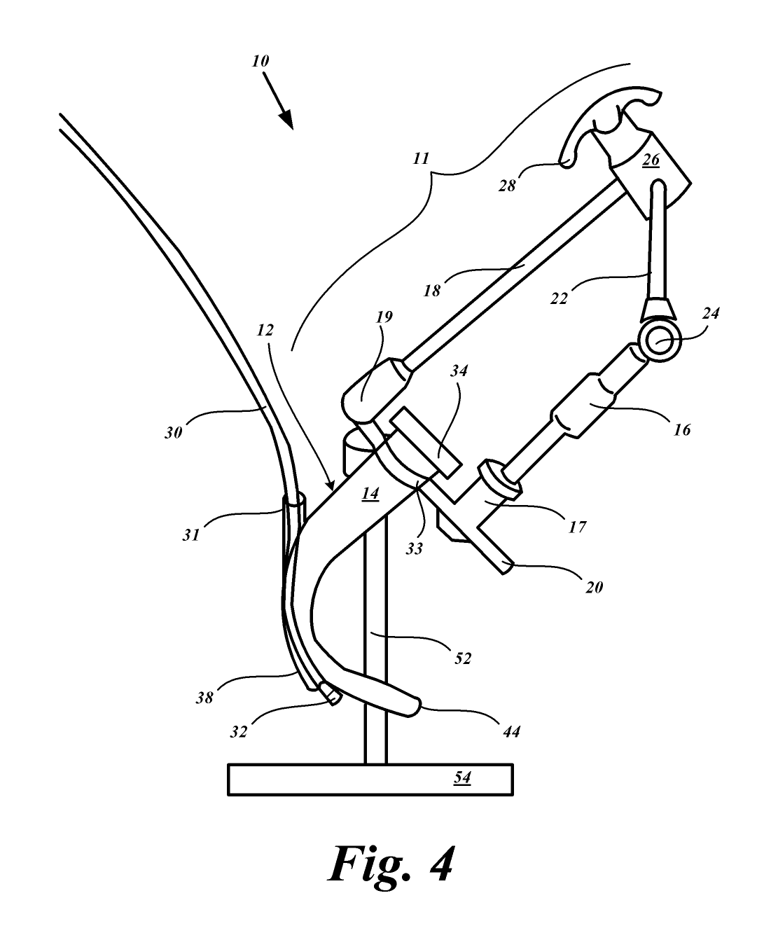

FIGS. 1-3 describe design and operational aspects of a non-transparent laryngoscope not having a obtuse angled entry and exit ports of the substantially transparent embodiments described in FIGS. 4-10B.

With regards to the U-shaped laryngoscope, FIG. 1 schematically depicts a U-shaped laryngoscope 25 that is not transparent and is lacking the ergonomic advantages of the transparent laryngoscope utilized in the surgical platforms illustrated in FIGS. 4-10B below. Here the fiber optic scope 15 is connected via fiber optic cable 30 to the U-shaped Laryngoscope 25 via fiber optic cable port 27.

FIG. 2 schematically depicts a laser fiber optic viewing through a camera apparatus through the non-transparent U-tube laryngoscope of FIG. 1. Here a medical user is crouched down uncomfortably near the level of operating room table 75 and is manipulating the fiber optic cable 30 via the fiber optic cable port 27.

FIG. 3 schematically depicts a bronchoscopic examination using the U-shaped laryngoscope of FIG. 1. Here a medical user is crouched down uncomfortably near the level of operating room table 75 and is manipulating the bronchoscope 15 that is connected with fiber optic cable 30 via the fiber optic cable port 27.

FIG. 3 schematically depicts a laser fiber optic viewing through a camera apparatus using the non-transparent U-shaped laryngoscope of FIG. 1. Here a medical user is crouched down uncomfortably near the level of operating room table 75 and is manipulating the laser 78.

FIGS. 4-10B below illustrate embodiments of a multipurpose single use surgical platform and intubation apparatus for ENT observation or surgery with a lifting bar handle, video scope passage and multiple operating channels. The surgical platform may have a number of configurations and methods of assembly with an operating stage that allows delivery of the relevant observation and/or surgical instruments reliably and at an angle that reduces the mechanical force required to obtain the access needed for diagnosis and therapy. The single-use surgical platform and intubation apparatus allows for many different kinds of procedures to be performed, including, for example, laser surgery, smoke evacuation and jet ventilation.

Embodiments described below include a single use intubation apparatus for placing an endotracheal tube in a patient. The intubation apparatus includes a scaffold having a plurality of moveable members, and a transparent laryngoscope attached to the scaffold and positionable by manipulation of the scaffold's moveable members. The transparent laryngoscope includes a handle region, a blade region, and a curved middle region between the blade and handle region. The blade region is configured to engage the tongue. Coursing from the handle region through or by the middle region and the blade region is at least one channel having a handle entry port and a blade exit port. Observation and surgical instruments can be inserted into this at least one channel, including for example fiberoptic bronchoscopes, laser surgery fiber optics, vacuum lines, jet ventilation instruments, and/or other surgical and/or observational instruments. Instruments exit the port on or near the blade to provide viewing and/or surgical actions, and/or to remove obliterated tissue fragments and steam, to provide ventilation to a patient, or for other purposes. Lighted lenses having an anti-fogging device to remove breath condensation or steam condensation from therapeutic laser procedures may also be included with the device. Heretofore described embodiments include the variable laryngoscope blade sizes available in the ENT GlideScope® product line which offer the enhanced utility of differently sized laryngoscope blades for the differently sized anatomical regions that vary between patients. These embodiments also provide single-use options where disposability is advantageous to help contain the spread of diseases deemed detrimental to the public health.

Other embodiments of the scaffold attached to the transparent laryngoscope allow the re-positioning of the transparent laryngoscope to obtain a different view of and/or to convey instruments to the region of interest of the patient's anatomy via the transparent laryngoscope. The repositioning of the transparent laryngoscope may be effected by a lifting handle that is in pivotable and/or rotatable connection with the moveable members. The instrument conveyable via the transparent laryngoscope includes a jet ventilation catheter and a therapeutic laser.

Particular embodiments provide for an option to deliver jet ventilation using a laser aimed jet ventilation catheter which has a fiber optic portion which may be central or peripheral and conducts light down the catheter in such a way that the light exits at the tip and serves as a visual guide for the anesthesiologist to aim the jet past the vocal cords in a way that will not entrain particles of tissue from the operating site but will provide a flow and pressure increase in the trachea and enable oxygenation and ventilation.

The laser treatment may include a dedicated laser fiber designed to channel a therapeutic laser through a channel and control it for treatment. The laser channel is angled to conform to the patient's airway anatomy.

The ear-nose-throat (ENT) surgical platform can include a camera or HDTV video laryngoscope, a protective lifter arm, protective supports on the sides of the arm which prevent unwanted secretions from clouding the lens, and a progressively curved middle section to the tongue blade which is united to a curved and slender handle portion which has a bar for attachment to the arm connected to the operating table scaffold. The HDTV camera may be highly resolving and re-postionable to accommodate the anatomy of the patient undergoing diagnostic or surgical procedures and to be ergonomically adaptable to the attending medical staff. The progressively curvable blade may be disposable.

An embodiment of the above includes the GlideScope® video enabled ENT laryngoscope substantially similar to the intubation and surgical apparatus 10 as discussed in detail regarding FIG. 4 below. The intubation and surgical apparatus 10 includes a laryngoscope 12 having a handle element 14 and a blade element 44 to lift the tongue so that the video system visualizes glottic structures. The video system includes a HDTV Video Bronchoscope amenable to rigorous disinfection procedures, a lighting source, and ancillary tools which can be delivered via open channels or around the body of the device.

There are several advantages to the GlideScope® video enabled ENT laryngoscope over existing devices: It is capable of being used in conjunction with a variety of commercially available flexible bronchoscopes. It is capable of being used in conjunction with a variety of commercially available operating tools, including, but not limited to fiber optics for laser surgery, forceps, biopsy needles, and irrigation and vacuum lines. The shape further allows smaller forces to be used to gain access to the patient's airway anatomy, reducing the potential for injury to the patient. The design allows the ENT surgeon a variety of different configurations based on the needs of the procedure being performed, equipment available and personal preferences. An alternate embodiment allows for a disposable design of the operating platform so that the apparatus is substantially available and clean to permit sanitary use in other patients. Disposable transparent or clear plastic sheaths may be adapted to the various embodiments described herein to provide efficient execution of laryngoscopic procedures between patients.

The variable sizes and the disposable portions of provided by the ENT GlideScope® product line are amenable to reducing the spread of infectious diseases deemed critically important to the public health because essentially no parts that encounter direct patient contact are reused. The operating platform may have a number of configurations and methods of assembly but the principle remains the same. The operating stage allows delivery of the relevant observation and surgical instruments reliably and at an angle that reduces the mechanical force required to obtain the access needed for diagnosis and therapy. The ability to use medically approved laser systems and fiber optics greatly extends the utility and effectiveness of the device and allows treatment to be carried down to the level of the smaller bronchi while at the same time providing jet ventilation if this is required. Smoke evacuation is also desirable for many procedures and this feature is integrated into the disposable blade embodiment. Other embodiments include a reusable laryngoscope blade, as for example where it is desirable to have a camera attached to the undersurface of a conventional blade to foster teaching demonstrations side-by-side with conventional laryngoscopy so that the camera assembly serves as an adjunct to regular laryngoscopy.

The provision of a jet ventilation channel provides that the jet ventilation is capable of being aimed approximately within 3-4 mm of the conduit. This renders a steady aim to be confidently established by an attending anesthesiologist. The steady aim allows the easy observation of the direction of the positive pressure discharge to assure that high-pressure gas does not enter the tissues but rather entrains air to ventilate the trachea and minimize pressure buildup.

The GlideScope® laryngoscope system provides for a video enabled laryngoscope that conveys visual confirmation of airway anatomy during airway procedures. Alternate embodiments provide for a disposable shell assembly to sheath the video apparatus and to provide a disposable option. The disposable design strategy provides effective cover for the video system. The electronic package may be fully immersible for cleaning and have all of the features of the regular video Laryngoscope system so that its adoption into the medical theater does not require additional training procedures. The disposable shell assembly is configured to have structural strength and durability to withstand sanitizing procedures.

The utility of a disposable shell option is that it provides the possibility of having a number of blade options suited to differing applications and clinical tasks. The range includes obesity, pediatrics, persons of small stature, normal adults, training designs, and neonatal designs. Thus with one master video unit the shell size may be selected to suit the clinical situation.

A CCD or CMOS camera placed at or near a point of angulation of the blade at or near the midpoint provides for advantageous positioning of the camera at some distance from the glottic opening to allow a degree of perspective and wide angle viewing. The position of the camera may be specifically placed or otherwise have its location adjusted relative to the blade to provide a view desired by the laryngoscope user.

A heating element on or nearby the lens area acts as an anti-fog device to preserve the view when the exhaled moisture-laden air from the airway falls upon the cool lens inserted from the ambient air. This extends the utility of the intubation apparatus substantially by obviating the need for removing the apparatus from the patient for lens cleaning operations. In another embodiment, the laryngoscope is a specially sized smaller laryngoscope for pediatric use. Access and control can be extended to the laryngoscopes used for surgery on the upper and lower airways by use of video based or image capture designs.

FIGS. 4-10B illustrate various embodiments described above for use with the GlideScope® video enabled ENT laryngoscope.

FIG. 4 schematically depicts an intubation and surgical apparatus 10 having a scaffolding structure 11 holding a transparent laryngoscope 12 with a flexible bronchoscope optical cable 30. The scaffolding structure 11 of the apparatus 10 includes adjustable support arms 16 and 18 that are connected with the cross arm 20 via an adjustable connector 19 connecting with the support arm 18 and a cup 17 with the support arm 16. The support arm 16 articulates within the cup 17 extending from the cross arm 20 and with a pivot joint 24 in pivotable contact with the support arm 16 and a pivot arm 22. The cross arm 20 supports the connector 19 and the cup 17 via a support band 33 that holds the transparent laryngoscope 12. The cross arm 20 is secured to a support pillar 52 attached to a support base 54. The scaffolding structure 11 also includes an arm base 26 that is in articulating and pivotable connection with the support arms 16 and 18. The arm base 26 is connected with a lifting arm handle 28 that is maneuverable by an operator or medical user.

The transparent laryngoscope 12 includes a handle 14 that encloses an handle ridge 34 configured to provide a stabilizing structure to facilitate manual re-positioning of the laryngoscope 12, or as an anchor component for engagement with the scaffold structure 11. The laryngoscope 12 also includes a cable entry port 31 for receiving a fiber optic cable 30, a tongue blade 40 having a tongue blade end 44, a middle section 38 located between the tongue blade 40 and the handle 14, and a cable exit port 35 beneath the tongue blade 40. The support band 33 secures the handle member 14 near the laryngoscope handle ridge 34. The fiber optic cable 30 may be a bronchoscope cable or a fiber optic cable associated with a surgical laser. Yet other embodiments include the cable 30 housing an electronic optical chip having an integral camera, for example a CMOS chip located at end of the cable 30 emerging benath the tongue blade 40.

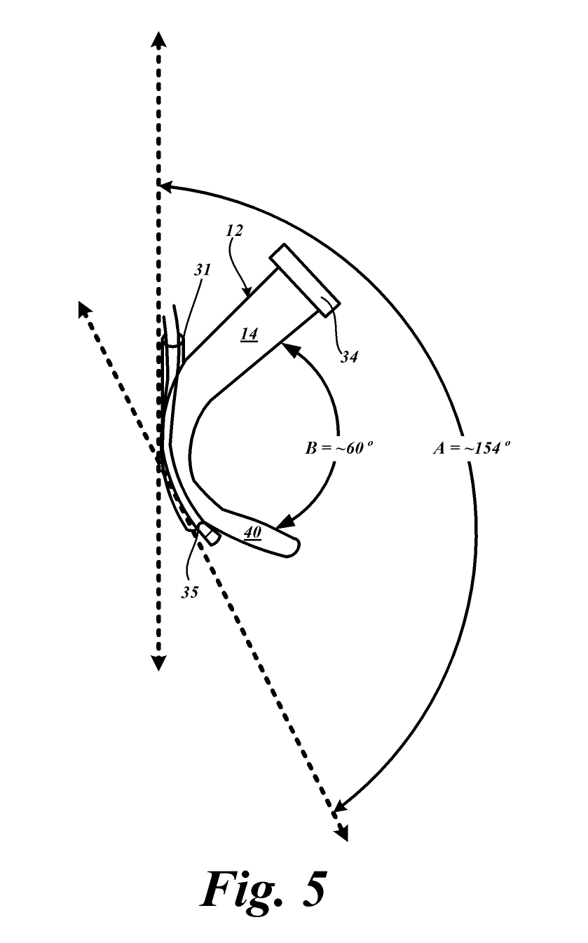

FIG. 5 illustrates the angle of inclination between the entrance and exit ports 31 and 35 and the handle 14 and blade 40 of an embodiment of the transparent laryngoscope 12. In this embodiment an obtuse angle-A of approximately 154 degrees between entrance and exit ports 31 and 35. The handle 14 is shown at an acute angle of inclination from tongue blade 40 of approximately 60 degrees is shown that separates the handle 14 from tongue blade 40. Other embodiments may have angle-A having obtuse angles other than 154 degrees and angle-B having a range of approximately 15 degrees to 90 degrees. The obtuse angle-A provides for the ergonomic positioning of a standing user relative to the substantially horizontal position of a bed or platform occupied patient.

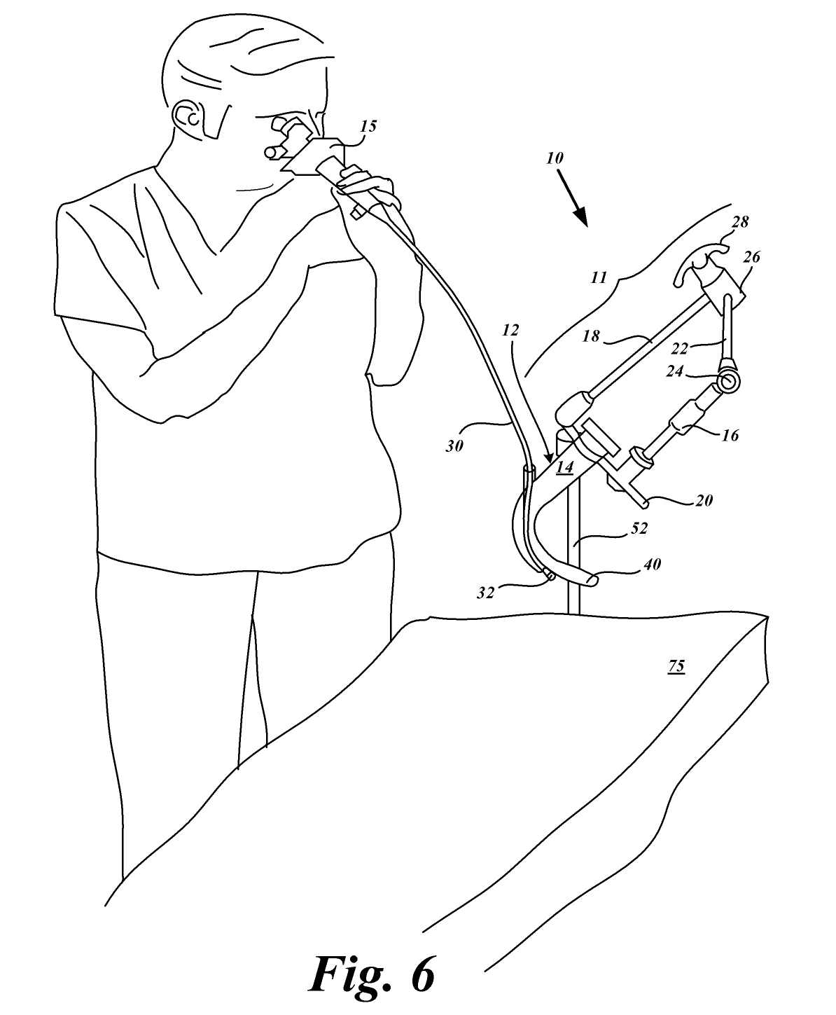

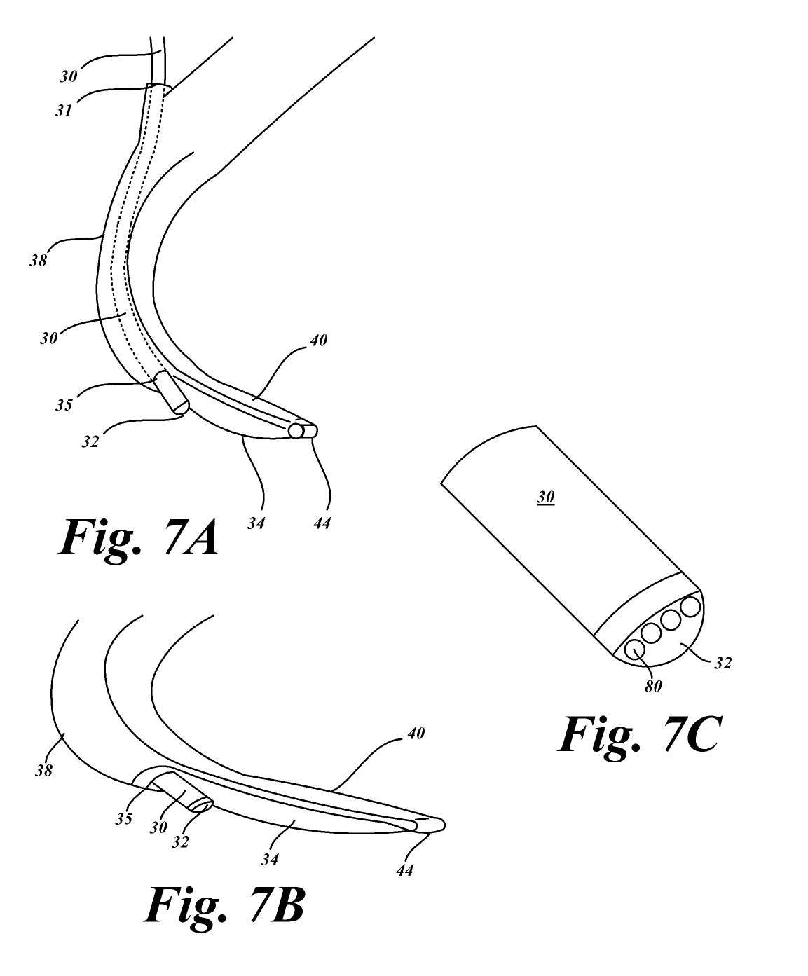

FIG. 6 schematically depicts an operator controlling a bronchoscope 15 passing through the auxiliary channel of the transparent laryngoscope 12 via bronchoscope fiber optic cable 30. The operator is able to stand and operate the bronchoscope 15 as routed through the transparent laryngoscope 12 in an ergonomic position. FIGS. 7A and 7B schematically depict a perspective side view of the tongue blade region of the transparent laryngoscope having different lengths and curvatures and showing a bronchoscope exiting the exit port beneath the tongue blade region. FIG. 7A schematically depicts the side view of the tongue blade 40 of the transparent laryngoscope 12 having a screen 32 or light and lens like structure at the terminal end of the fiber optic cable 30 emerging from the cable exit port 35 beneath the tongue blade 40. The fiber optic cable 30 is shown coursing through the middle section 38 between entry and exit ports 31 and 35. The screen 32 of the bronchoscope, connected via the fiber optic cable 30, is shown beneath and/or alongside the underside blade section 40 and retracted from blade end 44 to accommodate different viewing angles that are sufficient for executing laryngoscopic procedures for different patient anatomies. The position of the screen 32 relative to the exit port 35 may be altered by the user to accommodate desired viewing perspectives. A ridge 34 extending beneath the underside of the tongue blade 40 at approximately 90 degree is shown.

FIG. 7B illustrates the tongue blade region of the transparent laryngoscope 12 having a different tongue blade 40 length and different angle of inclination to the middle section 38 than the embodiment illustrated in FIG. 7A.

FIG. 7C illustrates a magnification of a particular embodiment of the screen 32 that caps the end of the fiber optic cable 30 emerging beneath the tongue blade 40 region of the embodiments illustrated in FIGS. 7A and 7B. The screen 32 has a central lens like region and a periphery of lights 80 to provide illumination to the patient's region of interest. The lights 80 may include light emitting diodes. Anti-fogging devices may be configured near the screen 32 to remove water condensation forming on the surface of the screen 32 arising from patient breathing and/or laser surgery procedures. The screen 32 and lights 80 may be includes a light source that is removeably attachable from the fiber optic cable 30.

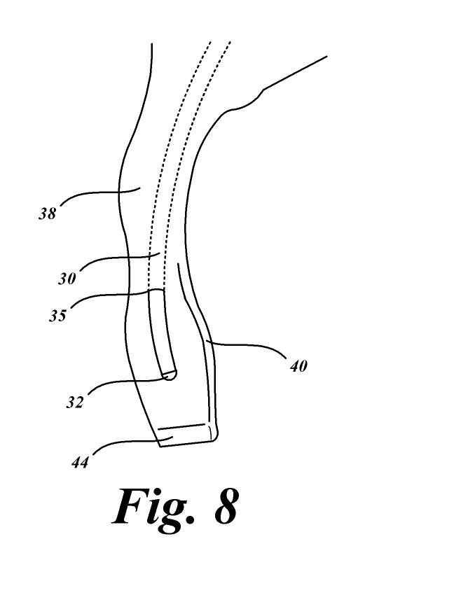

FIG. 8 schematically depicts a bottom view of the blade region 40 depicted in FIGS. 7A and 7B. The fiber optic cable 30 protrudes at a more pronounced length from the exit port 35 from the bottom portion of the blade region 40 than that depicted in FIGS. 7A and 7B. The screen 32 is exposed more substantially to provide another range of expansive viewings to the user. Here the screen 32 is shown closer to the blade end 44 to accommodate different viewing angles that are sufficient for executing laryngoscopic procedures for different patient anatomies.

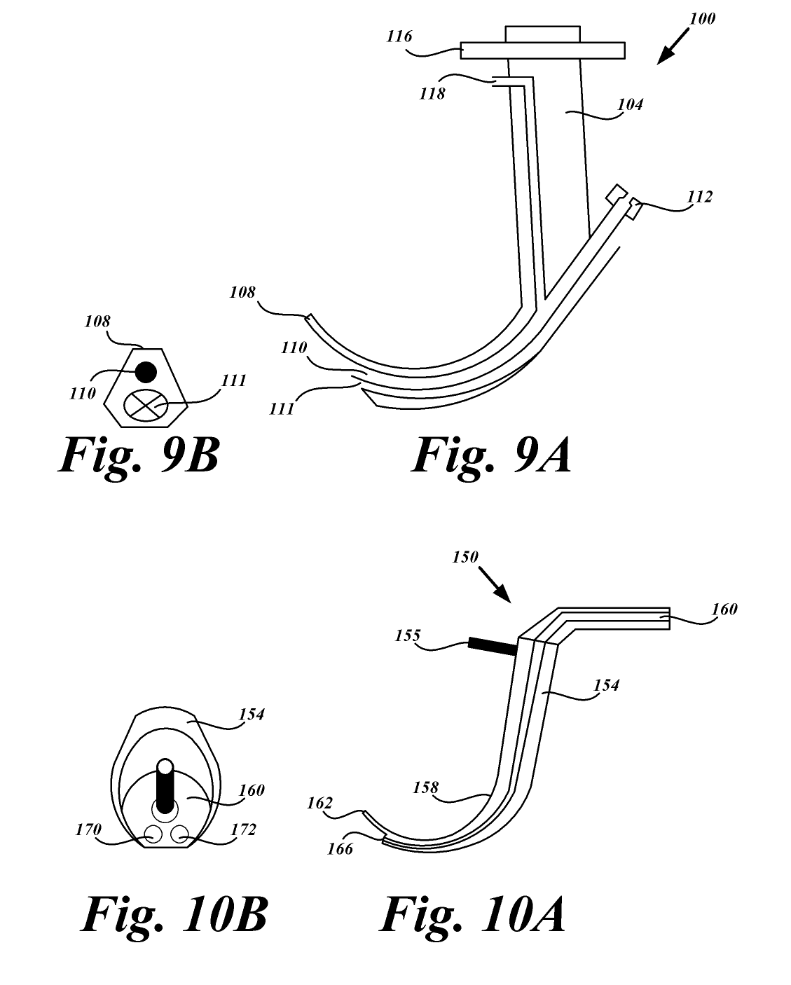

FIGS. 9A-B schematically depicts an ENT laryngoscope with a suction channel. FIG. 9A illustrates a cross section of an ENT suction laryngoscope 100. Laryngoscope 100 includes a handle 104 attached with a multiple-channel tongue blade 108 in which a vacuum channel 118 traverses through the interior of the handle 104 to the top channel 110 contiguous to video port 112 of the tongue blade 108. The patient side of the ports exiting channels 110 and 111 provide for a direct access view of the region of interest of the patient's anatomy.

FIG. 9B illustrates that the suction embodiment 100 doesn't require a separate port in that a midline channel is molded to form the top channel 110 and a lower channel 111. These double channels 110 and 111 are adjacent along the midline and provide for an easier manufacturing molding-based process. A rubber cap located nearby video port 112, in having access to the top channel 110, allows for the attachment of a video scope that helps stabilize the laryngoscope 100 and the blade 108 when engaged with the patient. The suction laryngoscope 100 may be configured in an external shell and contain a Bronchoscope, a laser fiber, and when attached to a suction source, provide suction ability. The blade 108 may comprise a progressive blade curvature to allow progressive blade entry and extended adjustability for a wide range of sizes. The curved lower portion of the handle 104 may also have a narrow configuration and thus be able to accommodate an extended range of throat anatomies. An operator may hold the ENT suction laryngoscope 100 at his end of the operation table via the handle 104 and secured by the handle ridge 116. Vacuum suction may be applied to the top channel 110 via the vacuum channel 118 to which video cable access are routed. In this arrangement the top channel 110 provides a two fold function, one function being to route video or electrical cables, and the other function providing a conduit for a vacuum source. The rubber cap located near the port 112 provides a sealable surface with any cables routing in the top channel 110 to preserve the vacuum conveyed to the top channel 110 via the vacuum channel 118.

FIGS. 10A-B schematically depicts in cross-section alternate embodiments of the transparent laryngoscope having a single use shell, wire cover, and an HDTV digital core. This alternate embodiment is an ENT laryngoscope 150 having a hard shell single-use handle region that is contiguous with a hard shell blade region 158 that includes a curving configuration to a blade tip 162. A holder 155 may be used as a securing bar for attachment with scaffolding 11 or for manual handling by a user. A high definition television (HDTV) digital core and wire 160 is shown traversing through the handle region 154, to the blade region 158, and to the viewing port 166 that is underneath and/or alongside and recessed from the blade tip 162. Alternate embodiments of the HDTV digital core include a high definition camera mounted to the end of the wire 160, wherein the high definition camera may be embedded within CMOS solid state circuitry to provide a three dimensional high definition image.

FIG. 10B illustrates that the handle 154 has three channels that are substantially triangularly arranged. A first channel is the HDTV digital core and wire 160 occupying the center region. A second channel 170 and a third channel 172 are located beneath the digital core and wire channel 160. The digital core and wire channel 160 can also be called a viewing channel 160. The second channel 170 can be used to convey illuminating light from a light source. The third channel 172 can be used to convey a vacuum from a vacuum source so that, for example, vapors from ablated tissue can be drawn away from the region of interest of the patient's anatomy. Alternatively, the second channel 170 can be used to convey both illuminating light from a light source and a vacuum from a vacuum source. In this and other alternative embodiments, the third channel 172 can convey other instruments, such as for example a therapeutic laser. In further embodiments, either one of the second channel 170 or third channel 172 can simultaneously convey both a surgical instrument such as a therapeutic laser and a vacuum. The patient side of the ports exiting channels 160, 170 and 172 provide for an instrument to view of the region of interest of the patient's anatomy.

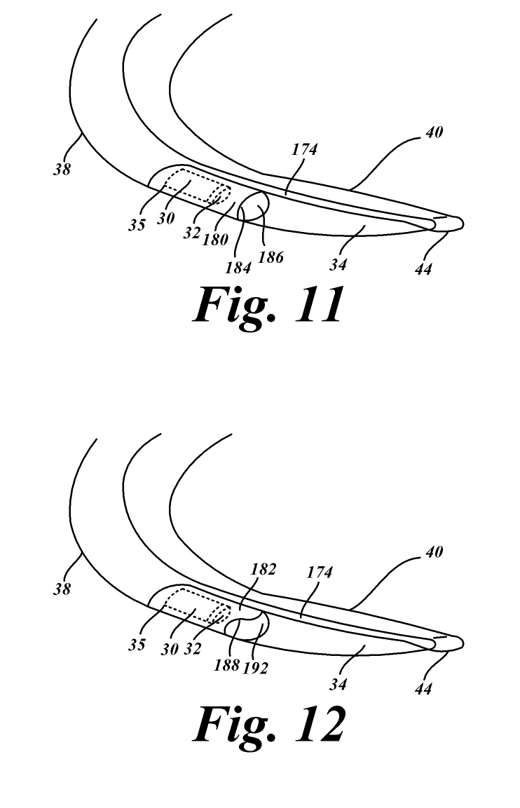

FIGS. 11 and 12 schematically illustrate different canopy embodiments that may be built into the laryngoscopes 12, 100, and 150 to enclose or envelop the lens to keep nearby tissue from obscuring the view of the screen 32. The tongue blade 40 provides a ledge 174 that serves as an overhang of protection or unbrella for the screen 32 against being obscured by blood, secretions, or aerosolized tissue or water vapor.

FIG. 11 illustrates a substantially cylindrical canopy 180 having a semicircular aperture wall 184 to define a substantially semicircular aperture 186. The cylindrical canopy 180 augments the protective and view preserving functions of the ledge 174 of the tongue blade 40. The canopy 180 also provides a tissue guard and skid function to smoothly assist insertion of the laryngoscopes 12, 100, and 150. The canopy 180 may also be transparent to maximize viewing angles.

FIG. 12 illustrates a substantially sinusoidal canopy 182 having a sinusoidal aperture wall 188 to define a substantially sinusoidal aperture 188. The sinusoidal canopy 182 augments the protective and view preserving functions of the ledge 174 of the tongue blade 40. The sinusoidal aperture wall 182 provides an increased viewing angle relative to the semicircular aperture wall 184 of FIG. 11. Similarly, the canopy 182 also provides a tissue guard and skid function to smoothly assist insertion of the laryngoscopes 12, 100, and 150. The canopy 182 may also be transparent to maximize viewing angles.

The laryngoscope with a disposable or reusable handle portion and lifter portion can have at or near its mid-portion an attached or inserted flexible video camera scope or high definition television (HDTV) to transmit to the operator the image from forward along the blade in a direction away from the handle and toward the tip and the airway. The laryngoscope can further have a disposable outer handle with multiple blade shell sizes and different angle configurations to allow an operator a choice of approaches to the airway in different anatomical situations, such as with different anatomical dimensions of individuals of differing age or for teaching reasons.

Yet other particular embodiments include a single-use laryngoscope having a handle area with a taper that permits advancement into the oral cavity, and a progressive curve which continues at a point of definition determined by a change in construction, texture or ridging form into the blade portion which has a continuous curvature. In this embodiment the laryngoscope is capable of continuous adjustment to adapt to various sizes of anatomy from small persons to large persons and the video camera or other viewer is located in the mid portion of the blade area to permit viewing around a corner, including at an angle ranging between 15 degrees and 90 degrees. The heater element of the single use laryngoscope may be removably connected to the flexible video scope to substantially heat the distal tip to approximately 40 degrees C.

The laryngoscope core may include a flexible geometry means to enable flexible adaptation to differing configurations of the shell. Other embodiments may provide for a light on the proximal or handle portion which trans-illuminates a translucent disposable laryngoscope shell to indicate the proper insertion depth by either a positive contact switch or by display of the text and graphic molded into the disposable part.

Another particular embodiment of the laryngoscope may include using a high quality video camera or a high definition television (HDTV) flexible video scope which may be removably attached to the undersurface of a U-shaped laryngoscope blade similar to those illustrated in FIG. 1 using a clip-on device having bars and a geometry that provides for removable attachment to an arm secured to the operating room or field use table. The clip on device provides an operating scope shell that operates as a hood or shield over the top of the electronic elements or other components routing through the laryngoscope. The components may attach to this clip on disposable shell device from beneath the laryngoscope so that the viewing elements are located in the zone of the oropharynx when the substantially transparent laryngoscope is fully inserted.

Other embodiments may include a channel or channels passing into the larynx area that may have a number of supportive channels dedicated from time to time to differing functions. The electronic assembly may comprise a rigid or flexible wire lead to a camera which has a heated lens, a light emitting diode (LED) lighting array, and a charge coupled device (CCD), or a complementary metal-oxide-semiconductor (CMOS) digital video camera for real time video monitoring of the airway for intubation and diagnosis. Other laryngoscope embodiments provide for connecting two bivalve halves to create an integrated whole unit that functions as a laryngoscope to enable visualization of the airway through a frontal portal permitting the passage of light and an uninterrupted image.

Particular embodiments of the laryngoscope may further include a retaining part or caps on the entry port for the laryngoscope that stabilizes the scope and prevents unwanted rotation or longitudinal motion and permits the scope channel to be used as suction. A suction channel joining one of the functional channels may be used to permit smoke evacuation. Other laryngoscope embodiments may include a channel or channels that allow the passing into the larynx area of a standard curve that will enable passage of either a flexible scope or a rigid special purpose scope. The laryngoscope may be configured to provide an aimable channel or channels passing into the larynx area that may be a dedicated channel for jet ventilation. Yet other embodiments of the laryngoscope may provide for shifting of Jet Ventilation channel from the right side to the left side as required to permit unobstructed viewing and treatment of the opposing side of the larynx.

While the preferred embodiment of the invention has been illustrated and described, as noted above, many changes can be made without departing from the spirit and scope of the invention. For example, the apparatus 11 may be configured to support the non-transparent laryngoscope 25 illustrated in FIG. 1 so that it may be re-positionable to obtain different views and/or convey instruments to the region of interest, including a jet ventilation catheter and a therapeutic laser. Accordingly, the scope of the invention is not limited by the disclosure of the preferred embodiment. Instead, the invention should be determined entirely by reference to the claims that follow.

The embodiments of the invention in which an exclusive property or privilege is: claimed are defined as follows:

1. An intubation apparatus for placing a tube by an operator to a region-of-interest comprising:
a transparent laryngoscope having a curved blade, a handle, and a middle section between the blade and handle;
at least one channel having a first port located on the handle and a second port located on the blade, the second port at an obtuse angle with the first port;
a fiber optic cable routeable from the first port through the second port;
a light source removeably attachable to the fiber optic cable emerging from the second port; and
a viewing device removeably attachable to the end of the fiber optic cable entering the first port,
wherein light is conveyed from the light source to the region of interest via the at least one first channel to provide a view of the region of interest.

2. The apparatus of claim 1, wherein the light source includes at least one of a light emitting diode, a laser, and a high emission incandescent source.

3. The apparatus of claim 2, wherein the at least one channel is configured to provide jet ventilation to the laser

4. The apparatus of claim 1, wherein the at least one channel is configured to convey vacuum from the region-of-interest through the transparent laryngoscope.

5. The apparatus of claim 1, wherein the at least one channel includes fiber optics.

6. The apparatus of claim 1, wherein the viewing device includes at least one of a television monitor, a computer monitor, a microscope, a video microscope, a CCD camera, a CMOS camera, and a high definition television.

7. The apparatus of claim 1, further including a scaffold attached to the transparent laryngoscope to allow the re-positioning of the transparent laryngoscope to obtain a different view of and convey instruments to the region of interest via the transparent laryngoscope.

8. The apparatus of claim 7, wherein the scaffold includes a plurality of moveable members.

9. The apparatus of claim 7, wherein the instruments conveyed via the transparent laryngoscope include a jet ventilation catheter and a therapeutic laser.

10. The apparatus of claim 8, wherein the plurality of moveable members includes a lifting handle to effect the re-positioning of the transparent laryngoscope.

11. A single use intubation apparatus for placing a tube by an operator to a region-of-interest comprising:
a scaffold having a plurality of moveable members;
a transparent laryngoscope positionable by the plurality of moveable members, the transparent laryngoscope having a curved blade, a handle, and a middle section between the blade and handle;
at least one channel having a first port located on the handle and a second port located on the blade, the second port separated by an obtuse angle with the first port;
a fiber optic cable routeable from the first port through the second port;
a light source removeably attachable to the fiber optic cable emerging from the second port; and
a viewing device removeably attachable to the end of the fiber optic cable entering the first port,
wherein light is conveyed from the light source to the region of interest via the at least one first channel to provide a view of the region of interest.

12. The apparatus of claim 11, wherein the viewing device includes at least one of a television monitor, a computer monitor, a microscope, a video microscope, a CCD camera, a CMOS camera, and a high definition television.

13. The apparatus of claim 11, wherein the scaffold includes a lifting arm handle to change the location of the plurality of moveable members.

14. The apparatus of claim 11, wherein the change of the location of the plurality of moveable members cause a change in the location of first, second, and third ports.

15. The apparatus of claim 14, wherein the third port is configured to convey instruments to the region of interest via the transparent laryngoscope.

16. The apparatus of claim 15, wherein the instruments conveyed to the region of interest via the third port of the transparent laryngoscope include a jet ventilation catheter and a therapeutic laser.
